(19) United States
(12) Patent Application Publication
(10) Pub. No.: US 2024/0102105 A1
(43) Pub. Date: Mar. 28, 2024

(54) BIOMARKER FOR PREDICTING RESPONSIVENESS TO ANTICANCER AGENT AND USE THEREOF

(71) Applicant: SAMSUNG LIFE PUBLIC WELFARE FOUNDATION, Seoul (KR)

(72) Inventors: Kyoung Mee KIM, Seoul (KR); Jee Yun LEE, Seoul (KR)

(21) Appl. No.: 18/270,307

(22) PCT Filed: Feb. 24, 2022
(86) PCT No.: PCT/KR2022/002687

§ 371 Date: Jun. 29, 2023

(30) Foreign Application Priority Data
Apr. 28, 2021 (KR) 10-2021-0055047

(51) Int. Cl. C12Q 1/6886; C12Q 1/6851; G01N 33/574

(52) U.S. Cl. CPC C12Q 1/6886; C12Q 1/6851; G01N 33/57492; C12Q 2600/106; C12Q 2600/158; G01N 2333/70596

(57) ABSTRACT

The present invention relates to a biomarker for predicting responsiveness to an anticancer agent and a use thereof, and more particularly, to: a marker composition for predicting responsiveness to atezolizumab, avelumab, or durvalumab, the marker composition comprising at least one gene selected from the group consisting of armadillo repeat-containing X-linked protein 1 (ARMCX1), serine/threonine-protein kinase D1 (PRKD1), and tyrosine kinase 2 (TYK2), or protein(s) encoded by the at least one gene; a composition for predicting responsiveness; a kit comprising the composition; and a method for providing information for predicting responsiveness. Gene markers according to the present invention are analyzed using patient-derived formalin-fixed paraffin-embedded tissues, and thus do not require the separate collection of samples, and the therefore convenient to analyze.

### TECHNICAL FIELD

The present invention relates to a biomarker for predicting responsiveness to an anticancer agent and a use thereof, and more particularly, to a marker composition for predicting responsiveness to atezolizumab, avelumab, or durvalumab, including one or more genes selected from the group consisting of armadillo repeat-containing X-linked protein 1 (ARMCX1), serine/threonine-protein kinase D1 (PRKD1), and tyrosine kinase 2 (TYK2), or a protein encoded by the gene, a composition for predicting responsiveness, a kit including the composition, and a method of providing information for predicting responsiveness.

### BACKGROUND ART

Cancer or a malignant tumor (malignant neoplasm) is a disease in which cells continue to divide because the cell cycle is not controlled, and is known to occur in any organ. Aggression and responsiveness to cancer treatment are known as reasons for the low survival rate of cancer patients, and since the reported data and the clinical outcome and prognosis of each patient are not always consistent, predicting the responsiveness and prognosis to a therapeutic in the anticancer treatment of a cancer patient provides the most suitable treatment method to cancer patients with different characteristics, and therefore toxicity related to unnecessary treatment can be avoided and a therapeutic effect may be ultimately enhanced.

In accordance with this necessity, recently, research on companion diagnostics referring to an approved diagnosis through which an appropriate target anticancer agent and therapeutic method can be selected based on the results of systematic analysis of individual parameters of each patient has been being actively conducted. Companion diagnostics can present a clear clinical basis for prescription according to a doctor's diagnosis, and can not only improve the efficiency of cancer treatment, but also contribute to the financial soundness of national health insurance by reducing misuse and abuse of a target anticancer agent. Currently, the companion diagnostics market is growing in the field of treatment of breast cancer, lung cancer, colorectal cancer, gastric cancer, melanoma, etc., and particularly, the breast cancer and lung cancer segments are expected to drive market growth. As pharmaceutical companies reduce the costs of developing new drugs and demands for target therapeutic agents increase, the global market for companion diagnostics was expected to grow 18% annually, reaching 5.8 billion dollars in 2019.

On the other hand, among anticancer agents, atezolizumab is a humanized immunoglobulin (Ig) G1 monoclonal antibody targeting PD-L1 that inhibits interaction between PD-L1 and PD-1 or B7-1 (known as CD80). In addition, avelumab is an immunoglobulin (Ig) G1 isotype complete human monoclonal antibody that selectively binds to PD-L1 and competitively blocks its interaction with PD-1. Compared with an anti-PD-1 antibody targeting T cells, avelumab is characterized by targeting tumor cells. In addition, durvalumab is a human immunoglobulin G1 kappa (IgG1k) monoclonal antibody forming a complex with PD-L1 that inhibits interaction of PD-1 (CD279) and a programmed death receptor 1 ligand (PD-L1). Like this, an anticancer agent inhibiting interaction with PD-1 by selectively binding to PD-L1 is used to treat a late solid tumor patient having a microsatellite unstable (MSI)/DNA mismatch repair-deficient (dMMR) biomarker.

Predictive biomarkers for immunotherapy differ from traditional biomarkers used in targeted therapy because of immune responses and the complexity of tumor biology. Recently, an immune-predictive score (IMPRES) based on 45 immune checkpoint genes was developed to predict a response to ICB in a melanoma patient (Nat Med. 2018 October; 24(10):1545-1549). Considering the necessity for a clinical-grade biomarker for guiding the selection of an agent for maximizing the difference in tumor biology and the potential for a patient benefit, the present inventors discovered a cancer-specific gene expression set for predicting responsiveness to atezolizumab, avelumab, or durvalumab.

### DISCLOSURE

### Technical Problems

To discover a gene biomarker for predicting responsiveness to a cancer immunotherapeutic agent, the present inventors conducted real-time reverse transcription PCR (RT-PCR) analysis by extracting RNA from a formalin-fixed paraffin-embedded tissue derived from a cancer patient who had received treatment with a PD-1 inhibitor, such as atezolizumab, avelumab, or durvalumab, and analyzed genes that were expressed differentially according to the responsiveness of the cancer patients, and based on this, completed the present invention.

Therefore, the present invention is directed to providing a marker composition for predicting responsiveness to an anticancer agent, including one or more genes selected from the group consisting of armadillo repeat-containing X-linked protein 1 (ARMCX1), serine/threonine-protein kinase D1 (PRKD1), and tyrosine kinase 2 (TYK2), or a protein encoded by the gene, wherein the anticancer agent is one or more selected from the group consisting of atezolizumab, avelumab and durvalumab.

In addition, the present invention is directed to providing a composition for predicting responsiveness to an anticancer agent, which includes an agent for measuring the level of mRNA of one or more genes selected from the group consisting of armadillo repeat-containing X-linked protein 1 (ARMCX1), serine/threonine-protein kinase D1 (PRKD1), and tyrosine kinase 2 (TYK2), or a protein encoded by the gene, wherein the anticancer agent is one or more selected from the group consisting of atezolizumab, avelumab and durvalumab.

In addition, the present invention is directed to providing a method of providing information for predicting responsiveness to an anticancer agent, which includes measuring the level of mRNA of one or more genes selected from the group consisting of armadillo repeat-containing X-linked protein 1 (ARMCX1), serine/threonine-protein kinase D1 (PRKD1), and tyrosine kinase 2 (TYK2), or a protein encoded by the gene in a biological sample derived from a subject, wherein the anticancer agent is one or more selected from the group consisting of atezolizumab, avelumab and durvalumab.

In addition, the present invention is directed to providing a method of predicting responsiveness to an anticancer agent, which includes measuring or detecting the level of one or more genes selected from the group consisting of armadillo repeat-containing X-linked protein 1 (ARMCX1; NCBI Accession No: NM_016608), serine/threonine-protein kinase D1 (PRKD1; NCBI Accession No: NM_002742), and tyrosine kinase 2 (TYK2; NCBI Accession No: NM_003331), or a protein encoded by the gene in a biological sample derived from a subject,
wherein the anticancer agent is one or more selected from the group consisting of atezolizumab, avelumab and durvalumab.

However, technical problems to be solved in the present invention are not limited to the above-described problems, and other problems which are not described herein will be fully understood by those of ordinary skill in the art from the following descriptions.

### Technical Solutions

To achieve the purpose of the present invention, the present invention provides a marker composition for predicting responsiveness to an anticancer agent, which includes one or more genes selected from the group consisting of armadillo repeat-containing X-linked protein 1 (ARMCX1; NCBI Accession No: NM_016608), serine/threonine-protein kinase D1 (PRKD1; NCBI Accession No: NM_002742), and tyrosine kinase 2 (TYK2; NCBI Accession No: NM_003331), or a protein encoded by the gene, wherein the anticancer agent is one or more selected from the group consisting of atezolizumab, avelumab and durvalumab.

In one embodiment of the present invention, the marker composition may further include a ubiquitin carboxy-terminal hydrolase L1 (UCHL1; NCBI Accession No: NM 004181) gene, or a protein encoded by the gene.

In addition, the present invention provides a composition for predicting responsiveness to an anticancer agent, which includes an agent for measuring the level of one or more genes selected from the group consisting of armadillo repeat-containing X-linked protein 1 (ARMCX1; NCBI Accession No: NM_016608), serine/threonine-protein kinase D1 (PRKD1; NCBI Accession No: NM_002742), and tyrosine kinase 2 (TYK2; NCBI Accession No: NM 003331), or a protein encoded by the gene, wherein the anticancer agent is one or more selected from the group consisting of atezolizumab, avelumab and durvalumab.

In one embodiment of the present invention, the composition may further include an agent for measuring the level of mRNA of a ubiquitin carboxy-terminal hydrolase L1 (UCHL1; NCBI Accession No: NM_004181) gene, or a protein encoded by the gene.

In addition, the present invention provides a kit for predicting responsiveness to an anticancer agent, which includes the composition.

In one embodiment of the present invention, the anticancer agent may be used to treat one or more carcinomas selected from the group consisting of gastric cancer, colorectal cancer, biliary tract cancer, lung cancer, skin cancer, head and neck cancer, Hodgkin's lymphoma, kidney cancer, and urothelial cell carcinoma.

In another embodiment of the present invention, the agent for measuring the mRNA level of the gene may be sense and antisense primers or probes, which complementarily bind to the mRNA of the gene.

In still another embodiment of the present invention, the agent for measuring the protein level may be an antibody specifically binding to a protein encoded by the gene.

In addition, the present invention provides a method of providing information for predicting responsiveness to an anticancer agent, which includes measuring the level of one or more genes selected from the group consisting of armadillo repeat-containing X-linked protein 1 (ARMCX1; NCBI Accession No: NM_016608), serine/threonine-protein kinase D1 (PRKD1; NCBI Accession No: NM_002742), and tyrosine kinase 2 (TYK2; NCBI Accession No: NM 003331), or a protein encoded by the gene in a biological sample derived from a subject, wherein the anticancer agent is one or more selected from the group consisting of atezolizumab, avelumab and durvalumab.

In one embodiment of the present invention, the method of providing information may further include measuring the level of mRNA of a ubiquitin carboxy-terminal hydrolase L1 (UCHL1; NCBI Accession No: NM_004181) gene, or a protein encoded by the gene.

In another embodiment of the present invention, the mRNA level may be measured by one or more kinds of methods selected from the group consisting of NanoString nCounter analysis, polymerase chain reaction (PCR), reverse-transcription polymerase chain reaction (RT-PCR), real-time PCR, RNase protection assay (RPA), microarray, and Northern blotting.

In another embodiment of the present invention, the protein level may be measured by one or more kinds of methods selected from the group consisting of western blotting, radioimmunoassay (RIA), radioimmunodiffusion, enzyme-linked immunosorbent assay (ELISA), immunoprecipitation, flow cytometry, immunofluorescence, Ouchterlony immunodiffusion, complement fixation assay, and protein chip assay.

In still another embodiment of the present invention, the biological sample may be cancer patient-derived tissue.

In yet another embodiment of the present invention, the tissue may be paraffin-embedded tissue.

In addition, the present invention provides a method of predicting responsiveness to an anticancer agent, which includes measuring or detecting the level of one or more genes selected from the group consisting of armadillo repeat-containing X-linked protein 1 (ARMCX1; NCBI Accession No: NM_016608), serine/threonine-protein kinase D1 (PRKD1; NCBI Accession No: NM_002742), and tyrosine kinase 2 (TYK2; NCBI Accession No: NM 003331), or a protein encoded by the gene in a biological sample derived from a subject, wherein

the anticancer agent is one or more selected from the group consisting of atezolizumab, avelumab and durvalumab.

In one embodiment of the present invention, the method may further include obtaining a biological sample from a subject or human patient.

### Advantageous Effects

The present inventors discovered a gene set for predicting responsiveness to an anticancer agent using a tissue sample derived from a cancer patient who had received a cancer immunotherapy with atezolizumab, avelumab, or durvalumab, and verified the efficiency of the markers using microarray data of the Asia Cancer Research Group and the RNA sequencing result in the Cancer Genome Atlas (TCGA) cohort. Therefore, a genetic marker according to the present invention is analyzed using patient-derived formalin-fixed, paraffin-embedded tissue, so it is not necessary to separately collect a sample, which is convenient for analysis. The genetic marker according to the present invention can predict the responsiveness to the cancer immunotherapeutic agent in advance and thus can provide information for selecting an optimal therapy, so it is expected to be effectively used in the clinical companion diagnostics field.

### BRIEF DESCRIPTION OF DRAWINGS

FIGS. 1a and 1B show the distinguishment of responsiveness to atezolizumab, avelumab, or durvalumab according to an IMAGiC score or a group, FIG. 1a shows that the cases of four groups, complete response (CR), partial response (PR), stable disease (SD) and progressive disease (PD), are divided into groups with high responsiveness, and FIG. 1b shows that the cases of four groups, complete response (CR), partial response (PR), stable disease (SD) and progressive disease (PD), are distinguished into responders and non-responders on the basis of the IMAGiC group.

FIG. 2 shows the result of analyzing the relationship between the IMAGiC model, a TMB level, microsatellite instability (MSI), and the PD.L1 score.

FIGS. 3a, 3b and 3c show the analysis of progression-free survival (PFS) according to the IMAGiC responsiveness, FIG. 3a shows the result of confirming PFS after treating patients with various types of carcinomas with anticancer agents, and FIGS. 3b and 3c show the results of comparing the differences in PFS and overall survival between the IMAGiC responder and a non-responder.

FIGS. 4a and 4b confirm the relationship between MSI or TMB, and IMAGiC responsiveness, FIG. 4a shows the Boxplot result comparing the IMAGiC score between TMB subtypes, and FIG. 4b shows the Boxplot result comparing the IMAGiC scores between MSI subtypes.

### MODES OF THE INVENTION

The present inventors tried to conduct research to discover a genetic biomarker for predicting the responsiveness to a cancer immunotherapeutic agent, and thereby found ARMCX1, PRKD1, TYK2 and UCHL1 genes as biomarkers, and based on this, completed the present invention.

Therefore, to achieve the above purpose of the present invention, the present invention provides a marker composition for predicting responsiveness to an anticancer agent, which includes one or more genes selected from the group consisting of armadillo repeat-containing X-linked protein 1 (ARMCX1; NCBI Accession No: NM 016608), serine/threonine-protein kinase D1 (PRKD1; NCBI Accession No: NM_002742), and tyrosine kinase 2 (TYK2; NCBI Accession No: NM_003331), or a protein encoded by the gene, wherein the anticancer agent is one or more selected from the group consisting of atezolizumab, avelumab, and durvalumab.

In addition, the present invention provides a composition for predicting responsiveness to an anticancer agent, which includes an agent for measuring the level of mRNA of one or more genes selected from the group consisting of armadillo repeat-containing X-linked protein 1 (ARMCX1; NCBI Accession No: NM_016608), serine/threonine-protein kinase D1 (PRKD1; NCBI Accession No: NM_002742), and tyrosine kinase 2 (TYK2; NCBI Accession No: NM_003331), or a protein encoded by the gene, wherein the anticancer agent is one or more selected from the group consisting of atezolizumab, avelumab, and durvalumab, and a kit for predicting the responsiveness to the anticancer agent, which includes the composition.

In the present invention, as a marker for predicting responsiveness to an anticancer agent in the cancer patient, a ubiquitin carboxy-terminal hydrolase L1 (UCHL1; NCBI Accession No: NM_004181) gene or a protein encoded by the gene may be further included.

The term “anticancer agent” used herein preferably refers to a cancer therapeutic agent inducing an anticancer effect by stimulating an immune system as a cancer immunotherapeutic agent, and in the present invention, more preferably a PD-1 antagonist, which may be atezolizumab, avelumab or durvalumab.

The term “antagonist” used herein refers to a material that acts antagonistically on the binding of a certain bioactive material to a receptor, but does not itself exhibit physiological actions through each receptor.

Among immunotherapy with the cancer immunotherapeutic agent, passive immune therapy is a treatment method of attacking cancer cells by injecting immune response components produced in large quantities ex vivo, such as immune cells, antibodies, or cytokines, to a cancer patient, and active immunotherapy is a treatment method of attacking cancer cells by actively activating or producing individual antibodies and immune cells. The present invention relates to a biomarker for predicting responsiveness to atezolizumab, avelumab or durvalumab when treating a cancer patient therewith in such immunotherapy and a use thereof.

In the present invention, the anticancer agent may be used to treat one or more types of carcinomas selected from the group consisting of gastric cancer, colorectal cancer, biliary tract cancer, lung cancer, skin cancer, head and neck cancer, Hodgkin's lymphoma, kidney cancer, and urothelial cell carcinoma, but the present invention is not limited thereto.

“Predicting responsiveness to an anticancer agent” means predicting whether a patient will respond preferably or non-preferably to a cancer immunotherapeutic agent, predicting the risk of resistance to the anticancer agent, or predicting the patient's prognosis, that is, recurrence, metastasis, survival, or disease-free survival, after immunotherapy. The biomarker for predicting responsiveness to a treatment according to the present invention may provide information for selecting the most suitable immunotherapy method.

Agents for measuring an mRNA level of the marker gene for predicting responsiveness to an anticancer agent may be sense and antisense primers or probes which complementarily bind to mRNA, but the present invention is not limited thereto.

The term “primer” used herein refers to a short gene sequence that serves as the starting point for DNA synthesis and refers to an oligonucleotide synthesized to be used in diagnosis or DNA sequencing. Primers may be used by being synthesized conventionally in a length of 15 to 30 base pairs, but the length may vary according to the use, and the primers may be modified by methylation or capping according to a known method.

The term “probe” used herein refers to a nucleic acid capable of specifically binding to mRNA in a length of several bases to hundreds of bases, produced by an enzymatic chemical isolation and purification or synthesis process. The presence or absence of mRNA may be confirmed by labeling a radioactive isotope, enzyme or fluorophore, and the probe may be designed and modified by a known method.

An agent for measuring a protein level may be an antibody specifically binding to a protein encoded by the gene, but the present invention is not limited thereto.

The term “antibody” used herein includes an immunologically specific antigen and an immunoglobulin molecule with responsiveness, and includes both of a monoclonal antibody and a polyclonal antibody. In addition, the antibody includes all forms produced according to genetic engineering, such as chimeric antibodies (e.g., a humanized murine antibody) and antibodies with different antigen-binding sites (e.g., a bispecific antibody).

The kit for predicting the responsiveness to an anticancer agent of the present invention may consist of one or more types of compositions, solutions or devices with different components suitable for an analysis method.

In another aspect of the present invention, the present invention provides a method of providing information for predicting responsiveness to an anticancer agent, which includes measuring the level of mRNA of one or more genes selected from the group consisting of armadillo repeat-containing X-linked protein 1 (ARMCX1), serine/threonine-protein kinase D1 (PRKD1), and tyrosine kinase 2 (TYK2), or a protein encoded by the gene in a biological sample derived from a subject, wherein the anticancer agent is one or more selected from the group consisting of atezolizumab, avelumab, and durvalumab.

The mRNA level may be measured by one or more kinds of methods selected from the group consisting of NanoString nCounter analysis, polymerase chain reaction (PCR), reverse-transcription polymerase chain reaction (RT-PCR), real-time PCR, RNase protection assay (RPA), microarray, and Northern blotting according to a conventional method known in the art, but the present invention is not limited thereto.

The protein level may be measured by one or more kinds of methods selected from the group consisting of western blotting, radioimmunoassay (RIA), radioimmunodiffusion, enzyme-linked immunosorbent assay (ELISA), immunoprecipitation, flow cytometry, immunofluorescence, Ouchterlony immunodiffusion, complement fixation assay, and protein chip assay according to a conventional method known in the art, but the present invention is not limited thereto.

The biological sample is cancer patient-derived tissue, and more preferably, includes tissue embedded in paraffin by fixing with a fixation solution such as formalin, but the present invention is not limited thereto.

In still another aspect of the present invention, the present invention provides a method of predicting responsiveness to an anticancer agent, which includes measuring or detecting the level of one or more genes selected from the group consisting of armadillo repeat-containing X-linked protein 1 (ARMCX1; NCBI Accession No: NM 016608), serine/threonine-protein kinase D1 (PRKD1; NCBI Accession No: NM_002742), and tyrosine kinase 2 (TYK2; NCBI Accession No: NM_003331), or a protein encoded by the gene in a biological sample derived from a subject,
wherein the anticancer agent is one or more selected from the group consisting of atezolizumab, avelumab, and durvalumab.

In the present invention, the method may further include obtaining a biological sample from a subject or human patient.

Hereinafter, the present invention will be described in further detail with reference to examples. These examples are merely provided to illustrate the present invention, and the scope of the present invention should not be construed as limited by the following examples.

### EXAMPLES

### Example 1. Experimental Preparation and Methods

1-1. Selection of Target Patients

In this example, to evaluate clinical responses to various anticancer agents (atezolizumab, avelumab and durvalumab), 89 patients classified by gender, type of carcinoma, cycle of immunotherapy, type of cancer immunotherapeutic agent, TMB level, MSI status, and PD-L1 CPS were analyzed, and divided into responders and non-responders as IMAGiC Groups as shown in Table 1 below.

TABLE 1

| | CR.PR | SD.PD | Total | |
|---|---|---|---|---|
| recist_bin | (N = 24) | (N = 49) | (N = 73) | p |
| Age (median & quartile | 64.0 [54.0; | 59.0 [52.0; | 61.0 [52.0; | 0.466 |
| range) | 71.0] | 67.0] | 70.0] | |
| Gender | | | | 0.457 |
| female | 10 (41.7%) | 26 (53.1%) | 36 (49.3%) | |
| male | 14 (58.3%) | 23 (46.9%) | 37 (50.7%) | |
| Cancer type | | | | 0.107 |
| cervix cancer | 0 (0.0%) | 1 (2.0%) | 1 (1.4%) | |
| cholangiocarcinoma | 3 (12.5%) | 7 (14.3%) | 10 (13.7%) | |
| colorectal cancer | 4 (16.7%) | 0 (0.0%) | 4 (5.5%) | |
| gastric cancer | 6 (25.0%) | 13 (26.5%) | 19 (26.0%) | |
| hepatocellular | 0 (0.0%) | 1 (2.0%) | 1 (1.4%) | |
| carcinoma | | | | |
| melanoma | 9 (37.5%) | 16 (32.7%) | 25 (34.2%) | |
| sarcoma | 0 (0.0%) | 5 (10.2%) | 5 (6.8%) | |
| urothelial carcinoma | 2 (8.3%) | 6 (12.2%) | 8 (11.0%) | |
| Treatment line of | | | | 0.948 |
| immunotherapy | | | | |
| 1 | 8 (33.3%) | 15 (30.6%) | 23 (31.5%) | |
| 2 | 8 (33.3%) | 19 (38.8%) | 27 (37.0%) | |
| ≥3 | 8 (33.3%) | 15 (30.6%) | 23 (31.5%) | |
| Immunotherapy regimen | | | | 0.668 |
| atezolizumab containing | 3 (12.5%) | 5 (10.3%) | 8 (11.0%) | |
| avelumab containing | 1 (4.2%) | 0 (0.0%) | 1 (1.43) | |
| durvalumab containing | 6 (25.0%) | 13 (26.4%) | 19 (26.0%) | |
| nivolumab containing | 4 (16.7%) | 11 (22.6%) | 15 (20.6%) | |
| pembrolizumab containing | 10 (41.6%) | 20 (40.7%) | 30 (41.1%) | |
| Number of immunotherapy | 14.0 [11.0; | 7.0 [3.0; | 9.0 [5.0; | <0.001 |
| cycle | 19.0] | 9.0] | 13.0] | |
| (median & quartile range) | | | | |
| Total TMB (median & | 7.0 [4.3; | 4.7 [3.1; | 5.5 [3.1; | 0.040 |
| quartile range) | 10.2] | 7.0] | 7.8] | |
| TMB | | | | 0.191 |
| High (≥10 mutations per | 6 (25.0%) | 6 (12.2%) | 12 (16.4%) | |
| megabase) | | | | |
| Low (<10 mutations per | 18 (75.0%) | 43 (87.8%) | 61 (83.6%) | |
| megabase) | | | | |
| MSI status | | | | 0.033 |
| MSI | 3 (12.5%) | 0 (0.0%) | 3 (4.1%) | |
| MSS | 21 (87.5%) | 49 (100.0%) | 70 (95.9%) | |
| PD-L1 CPS | 4.5 [1.0; | 0.0 [0.0; | 1.0 [0.0; | 0.001 |
| | 15.5] | 3.0] | 5.0] | |
| IMAGiC Group | | | | 0.005 |
| Non-responder | 17 (70.8%) | 47 (95.9%) | 64 (87.7%) | |
| Responder | 7 (29.2%) | 2 (4.1%) | 9 (12.3%) | |

1-2. RNA Extraction

To isolate total RNA from formalin-fixed, paraffin-embedded tissue, a tissue block derived from each cancer patient was cut into a fragment with a thickness of 4 μm. Afterward, an RNeasy FFPE kit (Qiagen, Germany) was used according to the manufacturer's instructions to isolate RNA. More specifically, the cut tissue was deparaffinized, treated with proteinase K, and a DNAse on a column was digested, followed by extracting RNA using RNase-free water. The total RNA sample isolated by the above method was stored at −80° C. before use, and an RNA concentration was measured using NanoDrop (Thermo Fisher Scientific, USA).

1-3. Verification using Quantitative RT PCR

To evaluate reproducibility of IMAGiC models according to the present invention using a different technical platform, conditions for quantitative real-time PCR performed with a reaction solution with a volume of 10 μL per reaction, including 5 μL of a 2× Taqman PreAmp Master Mix, 4 μL of a cDNA sample, and 1 μL of primers/probes in a 384-well plate using a 7900HT sequence detection system (Applied Biosystems, Foster City, CA, USA), were set. PCR amplification was conducted under the following conditions, and the same sample was amplified in three independent wells: repletion of 40 cycles at 50° C. for 2 minutes, 94° C. for 10 minutes, 95° C. for 15 seconds, and then 60° C. for 60 seconds.

1-4. Development of Model for Predicting Responsiveness to Anticancer Agent and Verification of Efficiency

To construct a model for predicting atezolizumab, avelumab or durvalumab, the mRNA expression level of a gene exhibiting a significant expression pattern and PD-L1 CPS of cancer tissue were analyzed using a linear regression model. In addition, MSI and TMB are known to be closely related to responsiveness to immunotherapy, and thus the results of the present invention were verified through analysis between subtypes.

1-5. Immunohistochemical Staining for PD-L1

Immunohistochemical staining (IHC) was performed using a representative section of each formalin-fixed, paraffin-embedded tissue (FFPE) sample. PD-L1 staining was performed using an FDA-approved monoclonal antibody, PD-L1 22C3 pharmDx (Dako, Carpinteria, CA). In addition, the results of IHC slides stained with PD-L1 were interpreted by a trained pathologist (KMK): CPS was evaluated according to the instructions for using PD-L1 IHC 22C3 pharmDx after summing the number of cells (tumor cells, lymphocytes, and macrophages) stained with PD-L1, dividing the result by the total number of surviving tumor cells, and multiplying the result by 100 (https://www.agilent.com/cs/library/usermanuals/public/29219_pd-l1-ihc-22C3-pharmdx-gastric-interpretation-manual_us.pdf). The PD-L1 IHC result was interpreted as positive when the score was 1 or more, and as negative when the score was less than 1.

### Example 2. Confirmation of Gene Exhibiting Significant Difference in Expression Response to Anticancer Agent

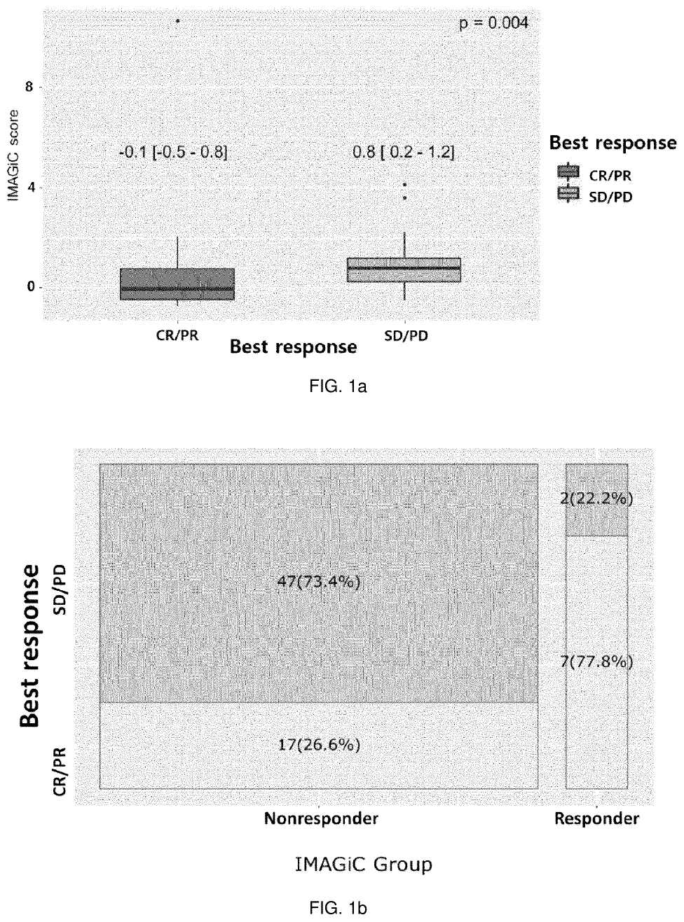

Gene expression profiling was analyzed from cancer tissues derived from 73 cancer patients in relation to the responsiveness to atezolizumab, avelumab or durvalumab. Based on the Response Evaluation Criteria in Solid Tumors (RECIST), the patients were divided into four groups, a complete response (CR), partial response (PR), stable disease (SD) and progressive disease (PD), according to the responsiveness to atezolizumab, avelumab or durvalumab, and as a result, as shown in FIG. 1A, in the group showing the best responsiveness, the IMAGIC score was analyzed as CR/PR(−0.1), SD/PD(0.8), and more specifically, as shown in FIG. 1B, the IMAGiC non-responders were divided into 47 cases of SD/PD (73.4%) and 17 cases of CR/PR (26.6%), and the IMAGiC responders were divided into 2 cases of SD/PD (22.2%) and 7 cases of CR/PR (77.8%).

In addition, according to the quantitative RT PCR, four genes, UCHL1, PRKD1, ARMCX1 and TYK2, exhibiting a significant difference in expression between the responders and the non-responders to the treatment with atezolizumab, avelumab or durvalumab were selected.

### Example 3. Construction of IMAGiC Model for Predicting Responsiveness to Anticancer Agent

To construct a model that can predict responsiveness to atezolizumab, avelumab or durvalumab, linear regression analysis was performed using mRNA expression levels of four genes selected from DEG. In addition, since the PD-L1 expression is an important biomarker for an atezolizumab, avelumab or durvalumab response, PD-L1 CPS was also used in IMAGiC.

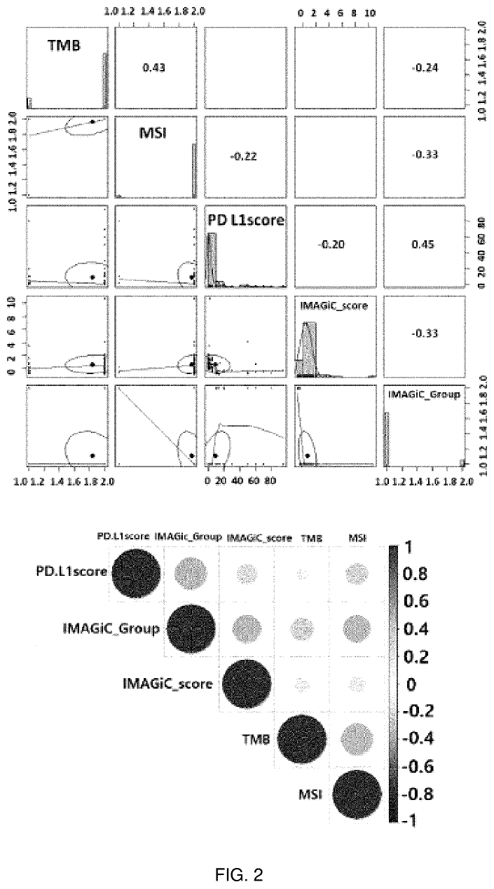

Finally, cancer patients were divided into responders and non-responders based on IMAGiC. As shown in FIG. 2, the IMAGiC groups had a correlation with a TMB group (r²=0.33) and MSI (r²=0.45).

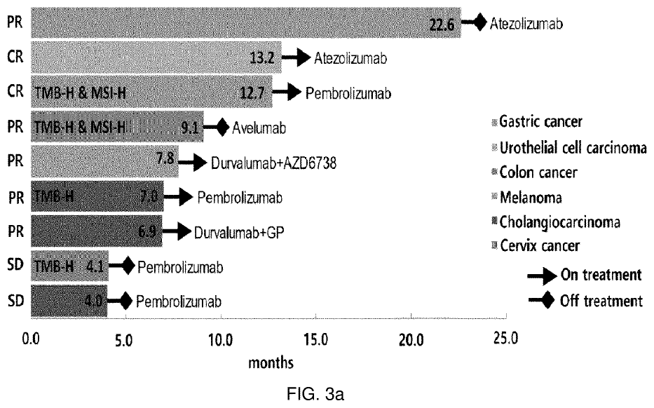

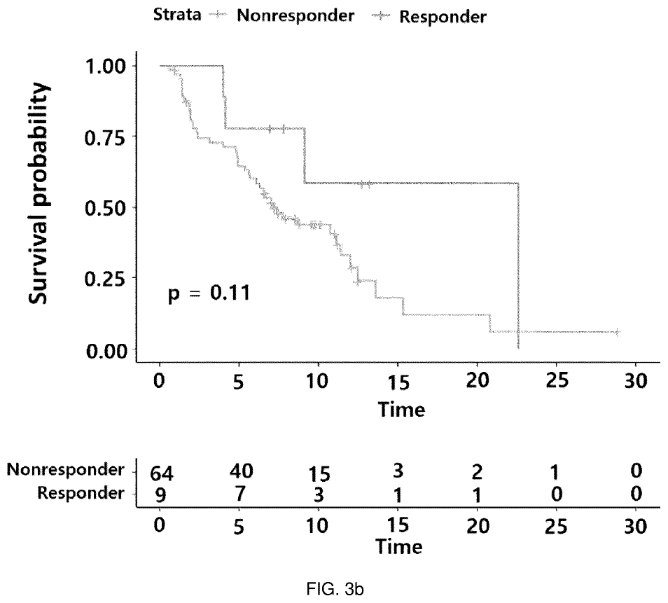

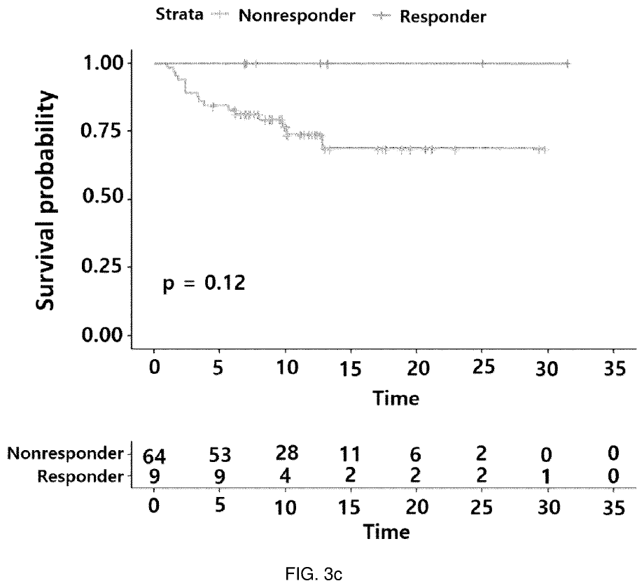

In addition, as a result of confirming PFS (month) according to the treatment of cancer patients with good IMAGiC responsiveness, and more specifically, patients with gastric cancer, urothelial cell carcinoma, colorectal cancer, melanoma, biliary tract cancer, and cervical cancer with atezolizumab, avelumab, durvalumab or pembrolizumab, as shown in FIG. 3A, it was confirmed that, when the gastric cancer patient was treated with atezolizumab, the patient showed a partial response (PR) for 22.6 months, when the urothelial cell carcinoma patient was treated with atezolizumab, the patient showed a complete response (CR) for 13.2 months, when the TMB-high and MSI-H gastric cancer patient was treated with pembrolizumab, the patient survived for 12.7 months, when the TMB-high and MSI-H colorectal cancer patient was treated with avelumab, the patient showed a partial response (PR) for 9.1 months, when the melanoma patient was treated with durvalumab and AZD6738, the patient showed a partial response for 7.8 months, when the TMB-high biliary tract cancer patient was treated with pembrolizumab, the patient survived for 7.0 months, when the biliary tract cancer patient was treated with durvalumab and GP, the patient showed a partial response for 6.9 months, when the TMB-high cervical cancer patient was treated with pembrolizumab, the patient showed progression-free survival for 4.1 months, and when the biliary tract cancer patient was treated with pembrolizumab, the patient showed progression-free survival for 4.0 months.

In addition, as a result of comparing the differences in PFS and overall survival between the IMAGiC groups, responders and non-responders, as shown in FIGS. 3B and 3C, the PFS medians of the responders and the non-responders were 22.6 months (95% CI 9.1˜) and 7.1 months (95% CI 5.712), respectively, and therefore the two responders can be clearly separated from the longer average value of PFS in the IMAGiC responder, and it was confirmed that the IMAGiC models are associated with PFS.

### Example 4. Analysis of Clinical Characteristics According to IMAGiC Responsiveness

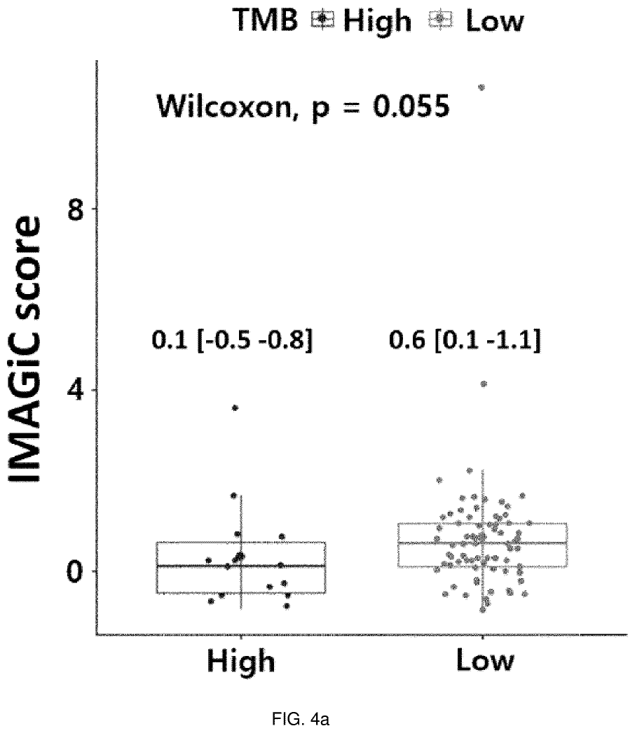

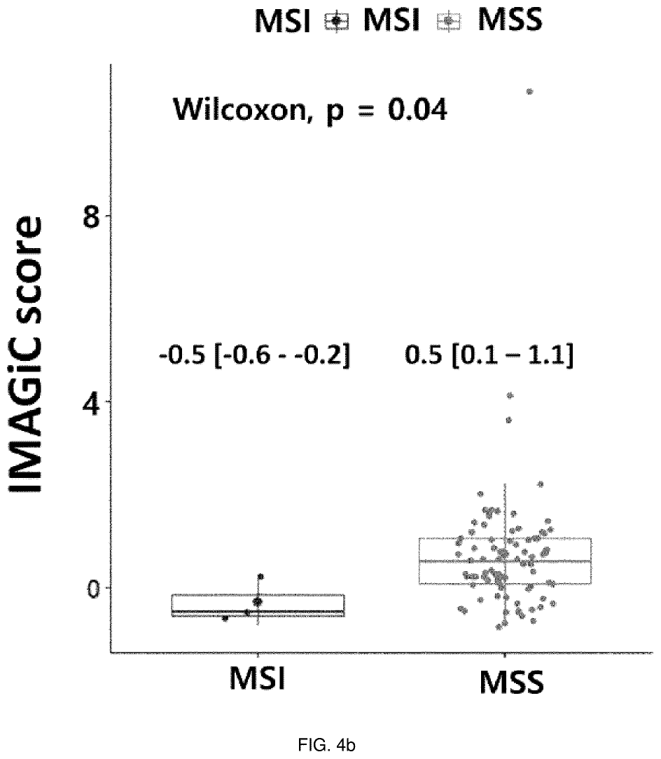

Since conventional PD-1 blockade has been reported to be effective on tumors not only with MSI but also with high ML, as a result of analyzing the relationship between IMAGiC and each subtype, as shown in Table 2 below, IMAGiC has a significant relationship with MSI (P=0.03) and TMB (P=0.03) statuses. That is, many patients with MSI or TMB High cancer were classified as a responder by IMAGiC. In addition, as shown in FIGS. 4A and 4B, it was confirmed that, in cancers associated with MSI and TMB, the IMAGiC score was significantly higher than those of microsatellite-stable (MSS) and non-TMB groups.

TABLE 2

| | Responder | Non-responder | |
|---|---|---|---|
| IMAGIG | (n = 10) | (n = 79) | p-value |
| Sex | | | 0.74 |
| Male | 4 (40.00%) | 39 (49.37%) | |
| Female | 6 (60.00%) | 40 (50.63%) | |
| Age | | | 1.00 |
| <60 | 5 (50.00%) | 38 (48.10%) | |
| ≥60 | 5 (50.00%) | 41 (51.90%) | |
| MSI | | | 0.03 |
| MSI-H | 2 (20.00%) | 1 (1.27%) | |
| MSS | 8 (80.00%) | 78 (98.73%) | |
| TMB | | | 0.05 |
| High | 4 (40.00%) | 10 (12.66%) | |
| Low | 6 (60.00%) | 69 (87.34%) | |
| PD-L1(CPS) | 32.5000 (±25.2730) | 5.7975 (±15.8255) | <0.001 |
| Mean numbers | 18.1100(±20.4630) | 11.8690 (±47.7927) | 0.13 |
| of SNV | | | |

SNV: single nucleotide variant

It should be understood by those of ordinary skill in the art that the above descriptions of the present invention are exemplary, and the example embodiments disclosed herein can be easily modified into other specific forms without changing the technical spirit or essential features of the present invention. Therefore, it should be interpreted that the example embodiments described above are exemplary in all aspects and are not limitative.

1.-15. (canceled)

16. A method for predicting responsiveness to an anticancer agent, comprising:
measuring the level of one or more genes selected from the group consisting of armadillo repeat-containing X-linked protein 1 (ARMCX1; NCBI Accession No: NM_016608), serine/threonine-protein kinase D1 (PRKD1; NCBI Accession No: NM_002742), and tyrosine kinase 2 (TYK2; NCBI Accession No: NM_003331), or a protein encoded by the gene in a biological sample derived from a subject, wherein the anticancer agent is one or more selected from the group consisting of atezolizumab, avelumab and durvalumab.

17. The method of claim 16, further comprising measuring the level of mRNA of a ubiquitin carboxy-terminal hydrolase L1 (UCHL1; NCBI Accession No:
NM 004181) gene, or a protein encoded by the gene.

18. The method of claim 16, wherein the anticancer agent is used to treat one or more carcinomas selected from the group consisting of gastric cancer, colorectal cancer, biliary tract cancer, lung cancer, skin cancer, head and neck cancer, Hodgkin's lymphoma, kidney cancer, and urothelial cell carcinoma.

19. The method of claim 16, wherein the mRNA level is measured by one or more kinds of methods selected from the group consisting of NanoString nCounter analysis, polymerase chain reaction (PCR), reverse-transcription polymerase chain reaction (RT-PCR), real-time PCR, RNase protection assay (RPA), microarray, and Northern blotting.

20. The method of claim 17, wherein the mRNA level is measured by one or more kinds of methods selected from the group consisting of NanoString nCounter analysis, polymerase chain reaction (PCR), reverse-transcription polymerase chain reaction (RT-PCR), real-time PCR, RNase protection assay (RPA), microarray, and Northern blotting.

21. The method of claim 16, wherein the protein level is measured by one or more kinds of methods selected from the group consisting of immunohistochemistry, western blotting, radioimmunoassay (RIA), radioimmunodiffusion, enzyme-linked immunosorbent assay (ELISA), immunoprecipitation, flow cytometry, immunofluorescence, Ouchterlony, complement fixation assay, and protein chip assay.

22. The method of claim 17, wherein the protein level is measured by one or more kinds of methods selected from the group consisting of immunohistochemistry, western blotting, radioimmunoassay (RIA), radioimmunodiffusion, enzyme-linked immunosorbent assay (ELISA), immunoprecipitation, flow cytometry, immunofluorescence, Ouchterlony, complement fixation assay, and protein chip assay.

23. The method of claim 16, wherein the biological sample is cancer patient-derived tissue.

24. The method of claim 23, wherein the tissue is paraffin-embedded tissue.
